(19) United States
(12) Patent Application Publication
(10) Pub. No.: US 2019/0178882 A1
(43) Pub. Date: Jun. 13, 2019

(54) IMMUNOASSAY-MULTIPLEXING APPARATUS

(71) Applicant: CELTEIN BIOSCIENCES, LLC, Monroe, OH (US)

(72) Inventors: Michael L. Jones, Monroe, OH (US); Aniruddha P. Puntambekar, Mason, OH (US)

(21) Appl. No.: 16/213,493

(22) Filed: Dec. 7, 2018

Related U.S. Application Data
(60) Provisional application No. 62/595,973, filed on Dec. 7, 2017.

(51) Int. Cl. G01N 33/543; B01L 3/00

(52) U.S. Cl. CPC G01N 33/54366; B01L 3/50855; B01L 2200/028; B01L 2300/161; B01L 2300/0829; B01L 2300/12; B01L 2200/0689

(57) ABSTRACT

An immunoassay-multiplexing apparatus includes a microplate having an array of cells. Each cell may have the same configuration, or different configurations. In at least one cell, there is a container within the cell. Within the container is a loading well and at least one satellite well. Each satellite well is connected to the loading well by a unique and independent interconnect channel. Each interconnect channel can taper inwardly transitioning from the associated satellite well to the loading well. Further, a sealing member is used to seal the cell.

### CROSS REFERENCE TO RELATED APPLICATIONS

This application claims the benefit of U.S. Provisional Patent Application Ser. No. 62/595,973, filed Dec. 7, 2017, entitled “IMMUNOASSAY-MULTIPLEXING APPARATUS”, the disclosure of which is hereby incorporated by reference.

### BACKGROUND

Various aspects of the present disclosure relate generally to an immunoassay-multiplexing apparatus, and to a process of loading an immunoassay-multiplexing apparatus.

Immunoassays are biochemical tests that are performed to detect and/or measure the existence of a particular molecule (e.g., proteins and hormones) in a sample through an identification marker. Types of materials that may be used as samples for immunoassays include serum, plasma, blood, urine, swabs, and various cultures. In some instances, multiplexing may be used to test the sample against multiple identification markers simultaneously, which may increase the overall speed of detection and/or measurement of the particular molecule.

### BRIEF SUMMARY

According to aspects of the present disclosure, an immunoassay-multiplexing apparatus is disclosed. The apparatus has a microplate with an array of cells. In this regard, a select cell within the array of cells has a container that contains a loading well, a satellite well, and an interconnect channel that couples the loading well to the satellite well. The loading well has an inferior inward taper. Moreover, the interconnect channel tapers inwardly as the interconnect channel transitions from the satellite well to the loading well. Further, the microplate includes a sealing member that seals the cell.

Each cell of the array of cells may have the same configuration, or one or more cells of the array of cells may have a different configuration. Moreover, in some embodiments, there can be more than one satellite well per cell, e.g., two satellite wells, four satellite wells, etc.

According to further aspects of the present disclosure, a process for loading an immunoassay-multiplexing apparatus is provided. The process comprises inverting an immunoassay-multiplexing apparatus, where the immunoassay-multiplexing apparatus comprises a microplate having an array of cells, and where at least one cell within the array of cells comprises a container. The container includes a loading well having an inferior inward taper, a first satellite well, a first interconnect channel, a second satellite well, and a second interconnect channel. The first interconnect channel couples the loading well to the first satellite well. Moreover, the first interconnect channel tapers inwardly as the first interconnect channel transitions from the first satellite well to the loading well. Analogously, the second interconnect channel (which is independent of the first interconnect channel) couples the loading well to the second satellite well. The second interconnect channel tapers inwardly as the second interconnect channel transitions from the second satellite well to the loading well.

The process further includes adding capture molecules to both the first satellite well and the second satellite well. In some embodiments, the capture molecules may be prevented from entering the loading well by the inward taper of the first interconnect channel and the second interconnect channel. The process also includes incubating the capture molecules in the immunoassay-multiplexing apparatus for a predetermined amount of time. The process yet further comprises aspirating the capture molecules, and then washing the first satellite well and the second satellite well. Further, the process includes adding a blocking buffer solution to the first satellite well and the second satellite well and incubating the blocking buffer in the immunoassay-multiplexing apparatus for a predetermined amount of time. Moreover, the process comprises aspirating the blocking buffer solution, and then washing the first satellite well and the second satellite well. Still further, the process comprises inverting the immunoassay-multiplexing apparatus so that the immunoassay-multiplexing apparatus is returned to its default orientation and sealing the immunoassay-multiplexing apparatus, e.g., with a hydrophilic sealing tape.

According to yet further aspects of the present disclosure, a process for analyzing an immunoassay-multiplexing apparatus is disclosed. The process comprises receiving a microplate having an array of cells, where at least one cell within the array of cells comprises a container. The container includes a loading well having an inferior inward taper, a first satellite (which may have a superior inward taper) well, a first interconnect channel, a second satellite well, and a second interconnect channel. The first interconnect channel couples the loading well to the first satellite well. Moreover, the first interconnect channel tapers inwardly as the first interconnect channel transitions from the first satellite well to the loading well. Analogously, the second interconnect channel (which is independent of the first interconnect channel) couples the loading well to the second satellite well. The second interconnect channel tapers inwardly as the second interconnect channel transitions from the second satellite well to the loading well. The container also has a sealing layer, e.g., of a hydrophilic sealing tape, disposed on the bottom of the microplate.

The process also comprises loading a sample material into the loading well, thereby distributing the sample material into the first satellite well via the first interconnect channel, and into the second satellite well via the second interconnect channel. The inward taper of the first interconnect channel prevents the sample material from re-entering the loading well from the first satellite well. Analogously, the inward taper of the second interconnect channel prevents the sample material from re-entering the loading well from the second satellite well.

The process further measuring an absorbance signal of the sample material in each of the first satellite well and the second satellite well at predetermined time intervals, calculating a trend based on the measured absorbance signal and time intervals, and identifying the sample material based on the trend.

### BRIEF DESCRIPTION OF THE SEVERAL VIEWS OF THE DRAWINGS

FIG. 1A is an example embodiment of an immunoassay-multiplexing apparatus according to various aspects of the present disclosure;

FIG. 1B is a partial view of an example embodiment of the apparatus of FIG. 1A, wherein the apparatus comprises a tray that receives separable arrays of cells according to aspects of the present disclosure;

FIG. 1C is an example array of cells usable with the apparatus of FIG. 1B according to aspects of the present disclosure;

FIG. 1D is an example illustration of the array of cells of FIG. 1C inserted into a row within the tray of FIG. 1B according to aspects of the present disclosure;

FIG. 2 is an example embodiment of a cell within the immunoassay-multiplexing apparatus of FIG. 1A according to various aspects of the present disclosure;

FIG. 3 is an alternate embodiment of the cell within the immunoassay-multiplexing apparatus of FIG. 1A according to various aspects of the present disclosure;

FIG. 4A is an illustrative example of the capillary stop effect used in the immunoassay-multiplexing apparatus according to various aspects of the present disclosure;

FIG. 4B is an alternate illustrative example of the capillary stop effect used in the immunoassay-multiplexing apparatus by including an indent in an interconnect channel passageway according to various aspects of the present disclosure;

FIG. 4C is yet another alternate illustrative example of the capillary stop effect used in the immunoassay-multiplexing apparatus by including multiple indents in an interconnect channel passageway according to various aspects of the present disclosure;

FIG. 5 is another alternate embodiment of the cell within the immunoassay-multiplexing apparatus of FIG. 1A according to various aspects of the present disclosure;

FIG. 6 is an example embodiment of a process for loading an immunoassay-multiplexing apparatus according to various aspects of the present disclosure;

FIG. 7 an example analysis set-up for analyzing an immunoassay-multiplexing apparatus according to various aspects of the present disclosure;

FIG. 8 is a graph of example absorbance data based on the analysis in FIG. 7 according to various aspects of the present disclosure; and

FIG. 9 is a process for analyzing an immunoassay-multiplexing apparatus according to various aspects of the present disclosure.

### DETAILED DESCRIPTION

Aspects of the present disclosure provide for the modification and improvement of technologies relating to immunoassay multiplexing. As disclosed herein, immunoassays are biochemical tests (e.g., an enzyme-linked immunosorbent assay (ELISA) test) that are performed to detect and/or measure the existence of a particular molecule in a sample (e.g., an analyte) through an identification marker (e.g., a capture antibody). Generally, these tests are performed in an apparatus referred to as a microplate.

One factor that may be significant to an entity performing biochemical tests via immunoassays is time. Each test or replicate requires a certain time commitment to complete the test. That time commitment may compound in a situation where multiple analytes need to be examined, or a high number of tests needs to be performed to ensure accuracy. One tool that may be used to mitigate that time commitment is a multiplex, which allows multiple analytes to be analyzed simultaneously. An immunoassay microplate using multiplex technology can take on a variety of form factors, where each form factor has a specified number of wells, such as a 96-well format, a 384-well format, or a 1536-well format, etc.

One benefit of a high number of wells (i.e., multiplexing) is that an entity can perform more tests or replicates simultaneously, thus saving time. However, larger well formats can require specialized and expensive machinery to automate the process, which generally limits the entities that can use the larger well formats to larger or well-funded labs.

Accordingly, aspects of the present disclosure are directed toward an immunoassay-multiplexing apparatus that allows for large scale testing using small scale techniques or machinery. Further, aspects of the present disclosure do not require specialized machinery or a requirement for personnel to learn a new process, thus allowing users to immediately the apparatuses as described herein.

An Immunoassay-Multiplexing Apparatus

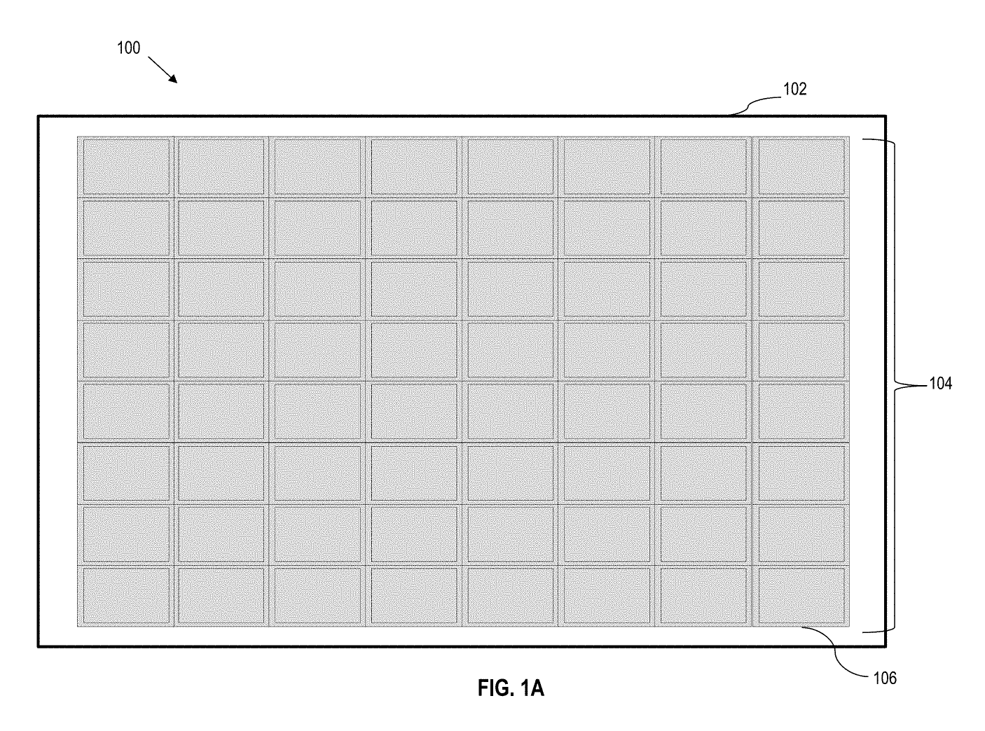

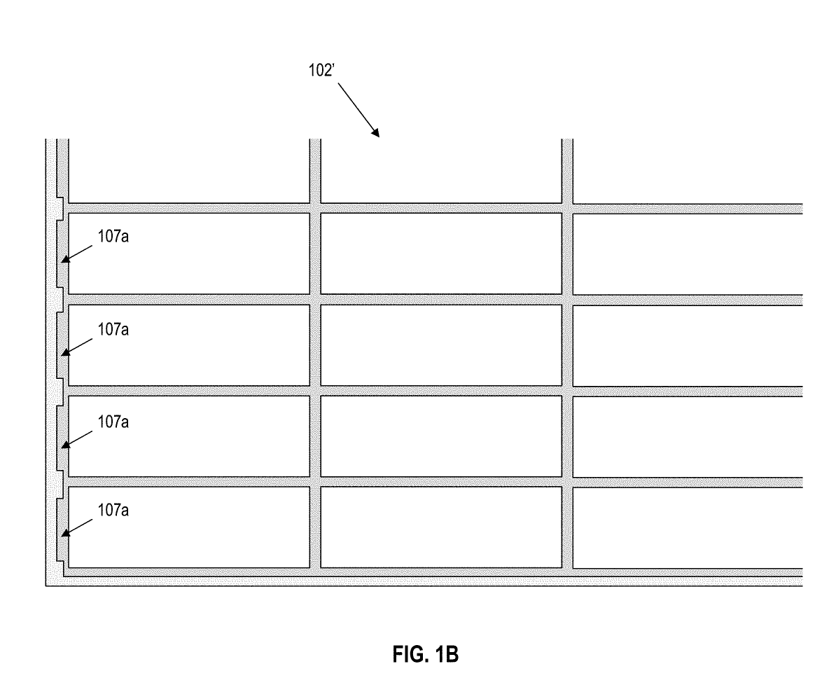

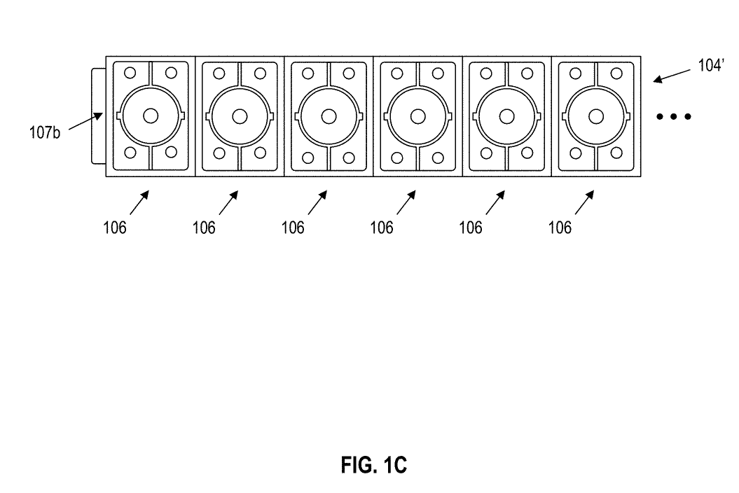

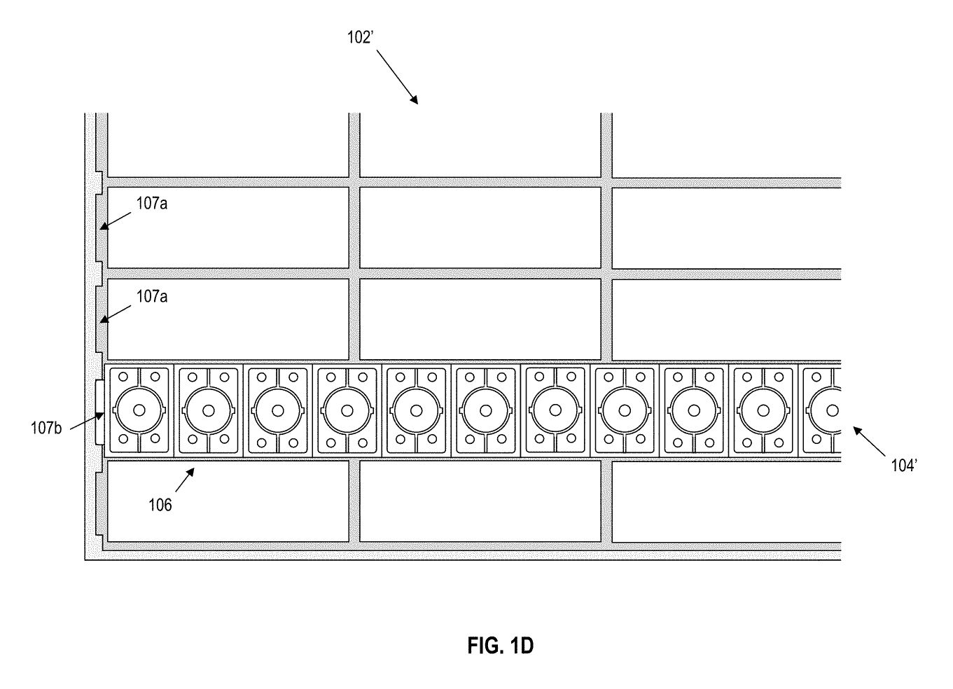

Referring to drawings and in particular FIG. 1A, immunoassay-multiplexing apparatus 100 is disclosed. The apparatus 100 comprises a microplate 102, which comprises an array 104 of cells 106. From a composition standpoint, many different materials may be used to construct the microplate 102, cells 106, and underlying structures. Examples of materials that are suitable include, but are not limited to, polystyrene, polycarbonate, Polymethyl methacrylate (PMMA), cyclic olefin copolymers (COC), combinations thereof, etc. Further, materials that are suitable include polymer materials that can be injection molded and exhibit moderate to high protein and nucleic acid adsorption. Moreover, hydrophobic variants of the aforementioned materials may be used. As described in greater detail herein, each cell 106 within the array 104 may vary in terms of structure (i.e., heterogeneous), or they can be uniform (i.e., homogeneous).

FIG. 1B is a zoomed in, partial view of an example embodiment of a microplate 102 shown in FIG. 1A. In FIG. 1B, the microplate 102′ is implemented as a tray having one or more rows capable of receiving an array (or arrays) of cells. Arrays can be a one-dimensional row of cells, a two-dimensional configuration comprised of rows of cells and columns of cells, etc. However, for purposes of illustration herein, the illustrated microplate 102′ is configured to receive rows of arrays. In this regard, the microplate includes a series of slots 107a disposed about an interior portion of the microplate 102′ (e.g., disposed about an interior portion of the perimeter of the microplate 102′). For example, the slots 107a may be positioned to extend longitudinally (i.e., lengthwise) along the microplate 102′ as shown in FIG. 1B. In this regard, the slots 107a provide structure to support the array of cells to minimize shifting during use. In this manner, each row can function to receive an array, align an array within the microplate 102′, lock or otherwise secure an array into the microplate 102′, combinations thereof, etc.

Referring to FIG. 1C, an example of an array 104′ suitable for use with the microplate 102′ of FIG. 1B is illustrated. The example array 104′ is comprised of a row of cells 106. The array 104′ also includes a tab 107b that is configured to mate with a corresponding slot 107a in the microplate 102′ (see FIG. 1B).

Referring to FIG. 1D, the microplate 102′ (FIG. 1B) is illustrated with an example array 104′ (FIG. 1C) installed in a row. The cells 106 of the array 104′ have been outlined with a thicker line weight for clarity of illustration.

Referring to FIG. 1A-FIG. 1D generally, the slots 107a are not restricted to extending longitudinally. For instance, the slots 107a may extend latitudinally (i.e., widthwise), longitudinally (lengthwise), or a combination thereof. Moreover, the slots 107′ can be on one end, on opposite ends, along multiple edges, etc., of each array 104′ depending upon the specific desired configuration.

Still further, in some embodiments, additional fastening mechanics can be implemented to secure the cells 106 within an array 104′ and/or to secure an array 104′ into a tray of a microplate 102′. Further, cells 106 within a given array 104 may be uniform, or heterogenous in composition.

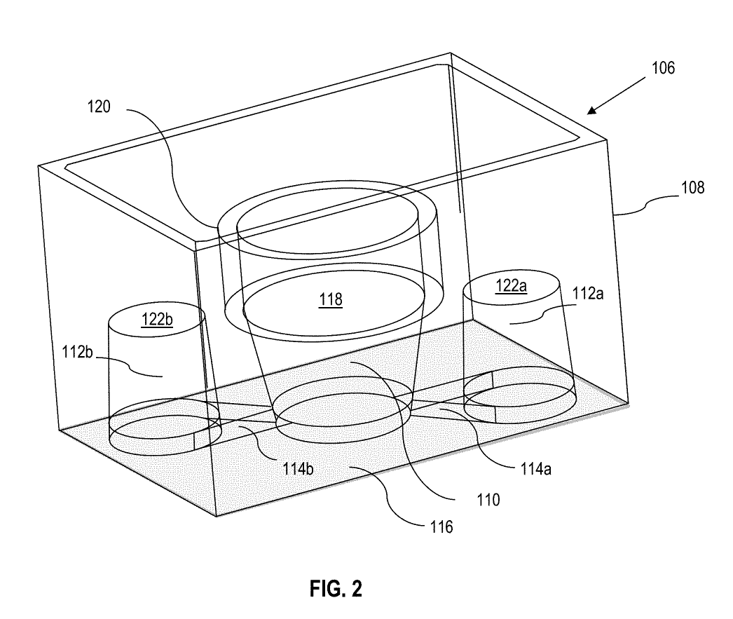

Now referring to FIG. 2, a select cell 106 is schematically illustrated according to aspects of the present disclosure. The select cell 106 can be utilized to implement one or more of the cells 106 within the array 104 of the apparatus 100 described with reference to FIG. 1A. The cell 106 of FIG. 2 can also be implemented as one or more of the cells of the array 104′ of FIG. 1C.

The select cell 106 comprises a container 108. The container 108 comprises a loading well 110 within the container 108. As illustrated, the loading well 110 has an inferior inward taper (i.e., the loading well 110 tapers inward as it extends downward within the container 108). Various shapes, sizes, and volumes for the loading well 110 may be used. For example, the loading well 110 may have a 5.5 millimeter (mm) diameter at the top, and 4 mm at the bottom with a 9 mm inward taper (e.g., center to center (CTC) spacing, which may be used throughout various embodiments herein).

The container 108 also comprises at least one satellite well, each satellite well coupled to the loading well 110 by an associated interconnect channel. For instance, in the example of FIG. 2, a first satellite well 112a is coupled to the loading well 110 by a first interconnect channel 114a. Analogously, a second satellite well 112b is coupled to the loading well 110 by a second interconnect channel 114b. In the illustrated example, the first interconnect channel 114a is independent of (e.g., distinct from) the second interconnect channel 114b. That is, in use, the first interconnect channel 114a can function as the sole fluid passageway from the loading well 110 into the first satellite well 112a. Likewise, the second interconnect channel 114b can function as the sole fluid passageway from the loading well 110 into the second satellite well 112b.

In the illustrated embodiment, the first interconnect channel 114a tapers inwardly as the first interconnect channel 114a transitions from the first satellite well 112a to the loading well 110. Likewise, the second interconnect channel 114b tapers inwardly as the second interconnect channel 114b transitions from the second satellite well 112b to the loading well 110.

For instance, an interconnect channel can be relatively more constricted proximate a transition from the loading well 110 into the interconnect channel relative to a portion of the interconnect channel located midpoint between the loading well and the corresponding satellite well. In this regard, the phrase “tapers inwardly as the interconnect channel transitions from the satellite well to the loading well” does not require a strict and constant narrowing along the entirety of the length of an interconnect channel. Rather, the taper can be along the entire length of an interconnect channel, or the taper can be along just a portion of the length.

In certain configurations, one or more of the interconnect channels can implement the taper using an extruded trapezoidal shaped geometry. However, other geometries may be utilized. Moreover, in some embodiments, one or more of the interconnect channels can be implemented as a microfluidic interconnect channel.

The container 108 can further comprise a sealing member 116 that seals the select cell 106. In various embodiments, the sealing member 116 is a hydrophilic sealing member. One example of an acceptable sealing member 116 is an adhesive sealing tape. In this regard, the sealing member 116 can be a single piece that extends across the entire microplate, or it may be applied to individual cells (or cell arrays, or cell strips).

The loading well 110 includes an inlet 118 that provides a means by which the loading well 110 can be loaded. In some embodiments, the loading well 110 includes an optional rim 120, which may aid in the loading of the loading well 110. Other features and benefits provided by the rim 120 are described in greater detail herein.

In some embodiments, satellite well(s) may also include an inlet, which provides a means to load an individual satellite well independent of the loading well 110. For instance, as illustrated, the first satellite well 112a can include a first inlet 122a, and analogously, the second satellite well 112b can include a second inlet 122b.

Although the select cell 106 can be implemented with a single satellite well 112, practical implementations can include multiple satellite wells 112. By way of example, in the illustrative implementation of FIG. 2, the container 108 comprises a first satellite well 112a within the container 108, and a first interconnect channel 114a that couples the loading well 110 to the first satellite well 112a. The interconnect channel 114a tapers inwardly as the interconnect channel 114a transitions from the first satellite well 112a to the loading well 110.

Likewise, the container 108 comprises a second satellite well 112b within the container 108, and a second interconnect channel 114b that couples the loading well 110 to the second satellite well 112b. Analogous to the description above, the second interconnect channel 114b tapers inwardly as the second interconnect channel 114 transitions from the second satellite well 112b to the loading well 108.

In various embodiments, the satellite wells 112 can have a superior inward taper as shown in FIG. 2, but such a taper is not required. Various shapes, sizes, and volumes for the satellite wells 112 may be used. For example, the satellite wells may have a 3.25 mm diameter at the bottom, and a 2.75 mm diameter at the top with a 4.5 mm inward taper.

In various embodiments, a container 108 can be implemented with a single satellite well with an associated interconnect channel. In other embodiments, such as that illustrated in FIG. 2, a container 108 can be implemented with two satellite wells, each satellite well with its own interconnect channel. In yet further embodiments, a container 108 can be implemented with other quantities of satellite wells and corresponding interconnect channels (e.g., 3, 4, or more). For instance, certain embodiments provide a container 108 with four satellite wells and corresponding interconnect channels.

Moreover, in select embodiments two or more satellite wells are equidistant from the loading well 110. In yet other embodiments, each satellite well is equidistant from the associated loading well. As such, in an embodiment with four satellite wells, each can be equidistant from the common loading well. Having each satellite well equidistant from the loading well may allow for more uniform distribution of materials from the loading well 110 to the satellite wells. A

Referring to FIGS. 1A-1D and FIG. 2 generally, each cell 106 within the array 104 of cells 106 can be dimensionally oriented in a variety of ways. For example, in one embodiment, each respective loading well 110 is oriented to conform to a conventional 96-well form factor in at least one direction. In other embodiments, or in combination with the 96-well form factor of the loading wells 110, each respective satellite well 112 is oriented to conform to a conventional 384 cell form factor in at least one dimension.

By way of example, according to certain aspects of the present disclosure, the apparatus 100 can be configured in the footprint of an SBS/ANSI compliant microplate combining elements of 96-well and 384-well microplate design. For instance, as shown in FIG. 2, each cell 106 can contain a loading well 110 (centrally located), which can be configured in a 96-well format. The cell 106 can also contain one satellite well 112, or a plurality of satellite wells surrounding the loading well 110. Within a standard 96-well microplate frame an embodiment can comprise 96 loading wells (in a 96-well format), each loading well with four satellite wells (in a 384-well format).

For instance, assume an example of four satellite wells 112 per cell 106. Under this configuration, in a practical example, for each cell 106, the loading well 110 can have a top diameter of 5.25 mm that tapers down to 4 mm. The cells are arranged such that adjacent loading wells 110 are arranged in a 9 mm pitch (96-well). Four satellite wells 112 are connected to each loading well via microfluidic interconnects.

In an example implementation, the satellite wells are 3.25 mm diameter at the base and taper up to 2.75 mm diameter and are arranged in a 4.5 mm pitch (to conform to a 384-well configuration). The plate bottom is sealed with a hydrophilic adhesive tape. Eight strips with 8×4 wells each allow for 64 distinct samples/standards to be tested in 4-plex (256 data points).

In an example configuration for a typical ELISA sequence, only capture molecules are added individually to the satellite wells (done as part of an ELISA kit manufacturing sequence). For end-user operation, all assay reagents and sample are added to the loading well 110 and are automatically divided to the satellite wells 112 as described more fully herein. Thus, in this example configuration, the apparatus 100 combines the ease of use of 96-well microplates and result density approaching a 384-well plate.

Specifically, for simultaneous singlet analysis of four analytes from the same sample, the apparatus 100 (implemented with cells 106, each having four satellite wells 112) allows for up to 600% improvement in the number of samples analyzed per run. More specifically, the apparatus 100 utilizes or accommodates both form factors. While certain embodiments comprise a 64 loading well/256 satellite well configuration, the microplate 102 can be scaled up or down depending on need (e.g., 32 loading wells/128 satellite wells, etc.). For instance, in various embodiments the cells 106 within the array 104 of cells 106 could be arranged in a strip-well format such that multiple strip-wells can be assembled within a frame for microplates. Each strip may contain, for instance, eight loading wells 110 and thirty-two satellite wells 112, which can be attached to an existing microplate 102 or used to create a new one.

In some embodiments, each cell 106 comprises a loading well 110, satellite wells 112, or combination thereof, which are implemented as through apertures. This allows loading from either side of the apparatus. However, a sealing member 116 (e.g., hydrophilic adhesive tape) can be used to cover the openings in the loading well 110 and/or satellite wells 112 in certain embodiments. Thus, a capture antibody solution and blocking buffer can be easily added to satellite wells 112 as part of a manufacturing sequence for ELISA kits (e.g., using liquid handlers). Once prepared, the apparatus is sealed using the sealing member 116.

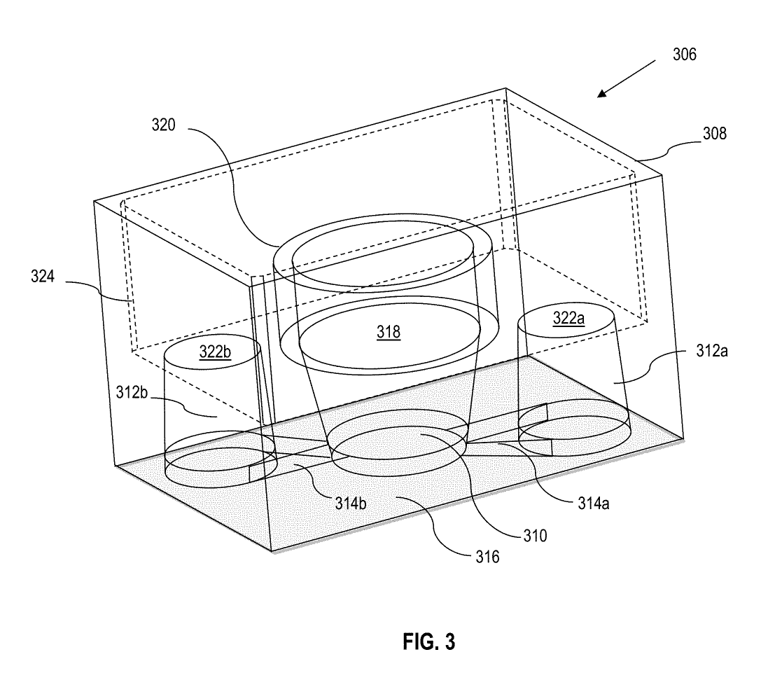

Now referring to FIG. 3, an alternate embodiment of a select cell 306 is schematically illustrated according to aspects of the present disclosure. The select cell 306 can be utilized to implement one or more of the cells 106 within the array 104 of cells 106 of the apparatus 100 described with reference to the preceding FIGURES.

As such, like elements are illustrated with like reference numbers two hundred higher than counterparts in FIG. 1A-FIG. 1D, and FIG. 2. Moreover, since the select cell 306 of FIG. 3 includes similar structures in many respects to the select cell 106 of FIG. 2, the description of such analogous structures, embodiments, or features are incorporated from FIG. 2 and is not further repeated, except where necessary to explain the differences.

FIG. 3 illustrates an embodiment where the container 308 further comprises a chamber 324 disposed above the satellite wells 312a and 312b. The chamber 324 surrounds the first inlet 318 for the loading well 310 and second inlets 322a, 322b for the satellite wells 312a, 312b, respectively. While only two satellite wells 312 are illustrated for visual clarity of the figure, the chamber 324 can be adapted to any number of satellite wells 312.

In the illustrative embodiment, the loading well inlet 318, as well as the satellite well inlets 322a, 322b are planar with a bottom surface of the chamber 324. However, the rim 320 of the loading well 310 extends within the volume of the chamber 324. The embodiment of FIG. 3 provides all loading capabilities described with reference to FIG. 2. Moreover, the embodiment of FIG. 3 enables loading all the satellite wells 312 simultaneously via the chamber 324.

Notably, the loading well 310 can be filled by means of access via the rim 320 and inlet 318. Moreover, the chamber 324 can be filled in a location outside the volume of loading well 310 defined by its rim 320, thus allowing each satellite well 312 to be filled (potentially simultaneously) with the same solution without also filling the loading well 310 by filling the chamber 324 below the rim 320.

Alternatively, filling the chamber 324 above the rim 320 allows the entire structure to be filled (i.e., loading well 310 and all satellite wells 312) in a convenient manner without requiring high precision alignment to each individual satellite well.

Alternatively, the embodiment illustrated in FIG. 3 may be geometrically defined as the chamber 324 (within the container 108) comprising the rim 120 of the loading well 310, whereas a first frustro-conical portion (defining the inferior inward taper) of the loading well 310 is below the chamber 324 in a manner analogous to the satellite wells 312. Further, the satellite wells 312 can each comprise a second frustro-conical portion defining a superior inward taper, and each interconnect channel 314 can comprise an extruded trapezoidal shaped channel between the loading well 310 and the corresponding satellite well 312.

In this regard, the first frustro-conical portion of the loading well 310 extends in a first dimension (i.e., downward in this example embodiment), and the second frustro-conical portion of the satellite well 312 extends oppositely in the first dimension spaced from the loading well (i.e., upward in this example embodiment). The extruded trapezoidal shaped channel of the interconnect channel extends in a second dimension orthogonal to the first dimension (i.e., laterally from the loading well 310 to the satellite well 312). Spatially, the chamber 324 extends over the loading well 300 and the satellite wells 312.

In various embodiments, the loading well inlet 318 is used to load a sample of interest (e.g., an analyte or other substrate), and the satellite well inlets 322a and 322b are used to load the identification markers (e.g., capture molecule).

Yet further, in some embodiments, the chamber 324 omits the satellite well inlets 322a and 322b, which would only leave access available to the loading well 310 via the loading well inlet 318. Such an embodiment may be useful for configurations where the identification markers (e.g., capture molecules) are already loaded in the apparatus (e.g., pre-loaded from a factory, or by a lab prior to the test). Such a configuration would prevent the sample of interest from inadvertently accessing the satellite wells 312a and 312b from the chamber 324.

Hybrid Configuration

The apparatus configurations in the various embodiments described herein provide numerous potential benefits. For instance, by using the hybrid configuration of the 96-well format combined with the 384-well format according to aspects of the present disclosure herein, a user of the apparatus 100 can obtain 256 test results from 64 unique samples (i.e., 64 loading wells, and 256 satellite wells) without any substantial change to the equipment/procedures. Further, these test results can be obtained by using machines and techniques that are used in 96-well format tests, thus potentially eliminating the need for more complex and expensive machines. With its hybrid configuration, the apparatus 100 can be used for absorbance, fluorescence, as well as chemiluminescence mode detection used with the ELISA platform.

Multiple Simultaneous Tests

Another benefit of the apparatuses and various embodiments thereof is the ability to perform multiple different types of tests within a single test session. As noted above, in some immunoassay tests, such as a test using a sandwich immunoassay format, are directed toward identifying a sample (e.g., an analyte) through an identification marker (e.g., a capture antibody and a detection antibody). In some circumstances, where multiple confirmations are needed to positively identify a particular analyte, each satellite well may be loaded with the same capture molecule.

However, in circumstances where it is preferable to utilize multiple different capture molecules to identify the particular analyze, each satellite well can be loaded with a different capture molecule (e.g., one different capture molecule in each of the four satellite wells, or two of the same capture molecules in two wells and two different capture molecules in the other, or three wells for capture molecules and one well for a control variable, etc.).

Reduced Cross-Reactivity

Yet another benefit of the apparatuses and various embodiments thereof relates to mitigation or elimination of cross-reactivity (or “cross-talk”). One potential issue with multiplex assays is the potential for cross-reactivity between pooled detection antibodies and capture antibodies specific to selected analytes. This leads to non-specific (spurious) signals, and undesirable results. One disadvantage of having structures such as loading wells coupled to structures like satellite wells is the possibility of back flow or back wash, which can result in cross-reactivity.

However, the apparatuses as disclosed herein does not have that disadvantage due to microfluidic dynamics imposed by the geometry of the apparatuses, as described more fully herein.

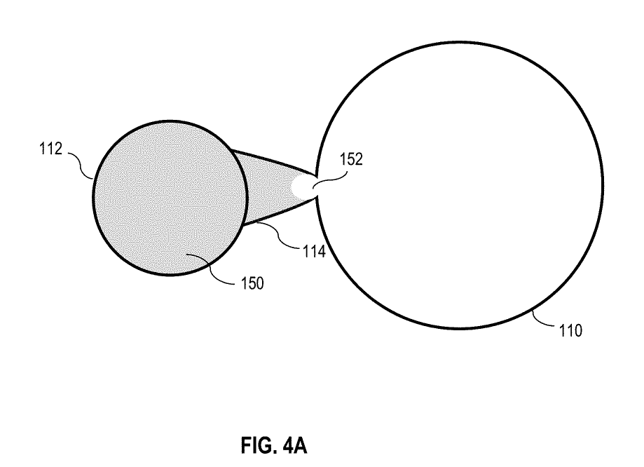

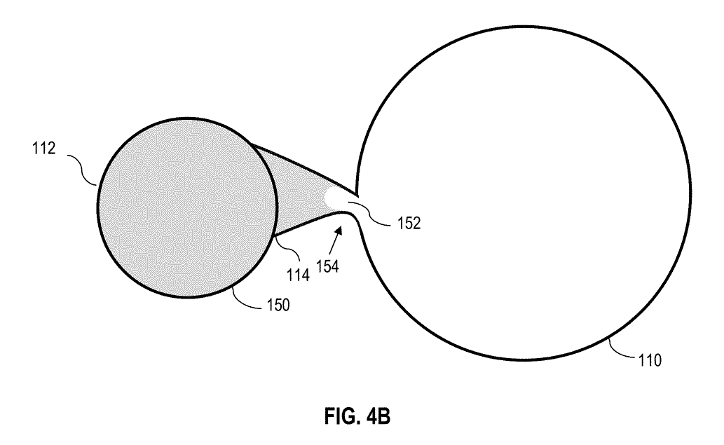

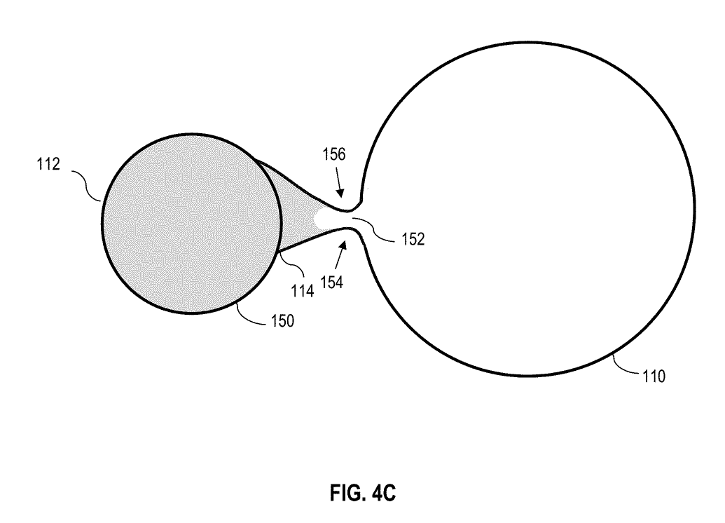

Referring to FIG. 4A, which is a close up view of a loading well 110 and an interconnect channel 114 of FIG. 2 (but may apply to FIG. 3 or any of the preceding FIGURES). Only one interconnect channel is shown for clarity of illustration. However, in practice, multiple interconnect channels can be implemented. Generally, when fluid 150 is introduced into open space, it will travel freely in the open space. In a scenario where cross-reactivity is a potential issue, it is not preferable for fluid 150 (e.g., the capture molecule) to freely travel where it wants to go. The apparatus 100 mitigates, or even eliminates the flow of fluid 150 from the satellite well 122 to the loading well 110 by using capillary stop action via a meniscus 152 of the fluid 150.

Generally, a meniscus is the curve in the upper surface of a liquid close to the surface of a container or another object, caused by surface tension. The meniscus of a liquid-solid interface is dictated by the liquid type and the surface energy of the solid it is in contact with. Given the right environmental factors, the surface tension of the meniscus can prevent a liquid from transitioning from one space to another, which is being leveraged by the apparatus 100.

As the fluid 150 attempts to enter the loading well 110 from the interconnect channel 114, the geometry of the interconnect channel 114 (i.e., the taper) in relation to the loading well tightens the meniscus 152, thus trapping the fluid 150 in the interconnect channel 114.

By virtue of that same geometric relationship, when the fluid 150 is added to the loading well 110, the fluid 150 will flow freely into the interconnect channel 114 and end up at the satellite wells 112. The sealing member 116, when used in a hydrophilic form also aids in moving the fluid 150 from the loading well 110 to the satellite wells 112.

As a result of the above, the apparatus 100 utilizes a completely passive mechanism to move fluid 150 (e.g., the sample of interest) from the loading well 110 to the satellite wells 112. However, in certain embodiments active mechanisms such as pumps, vacuums, or any mechanism suitable for moving liquid from the loading well 110 to the satellite well 112 may be incorporated.

In embodiments where the satellite well 112 has the superior inward taper, as the fluid 150 flows into satellite well 112 and begins to fill up the satellite well 112, the superior inward taper will create the same capillary stop effect that was generated at the interconnect channel/loading well interface. As the fluid 150 reaches the top of the satellite well 112, significant energy (differential pressure) is required for the fluid 112 to exit the satellite well 112 and an equilibrium condition is reached (i.e., stops the flow of the fluid 150). To further increase the capillary stop effect, the average diameter of the satellite well 122 can be made smaller, which will exhibit significantly higher capillary stop effect than larger diameter satellite wells 112.

In an example application where there are four identical satellite wells 112 (e.g., satellite wells 112a-d in FIG. 3), an ideal liquid handling situation would be where exactly four times (4×) a single satellite well 112 volume of fluid 150 is added to the loading well 110. As the fluid 150 is divided to the four satellite wells 112, the fluidic connection between the satellite wells 112 is broken and the potential for any cross-talk is virtually eliminated.

Moreover, further modifications can be made to the interconnect channel 114 to further reduce the potential of cross talk between the loading well 110 and the satellite well 112 (and corresponding interconnect channel).

Referring to FIG. 4B, an example embodiment is illustrated where the interconnect channel 114 has been modified by a first indent 154 on one side of the interconnect channel 114, thus causing the meniscus 152 to form further away from the loading well 110.

Referring to FIG. 4C, an example embodiment is illustrated where the interconnect channel 114 has been modified by a first indent 154 on one side of the interconnect channel 114 and a second indent 156 on another side of the interconnect channel 114, thus causing the meniscus 152 to form further away from the loading well 110.

Referring to FIG. 4A-4C, in practice, there need not be an indent. Alternatively, one or more indents can be provided, in various positions that are selected based upon the configuration of the interconnect channel to cause the meniscus 152 to form relatively further away from the loading well 110. In this regard, the exact indent positioning may vary depending upon the implementation to further increase benefits gained by the first indent 154 and/or the second indent 156.

Reduced Sample Consumption

Yet another potential benefit of the apparatus 100 is a reduced sample consumption per test. Rather than adding a sample (e.g., analytes) to each satellite well 112, as would be the case in a 384-well scenario, analytes are only added to the loading wells 110, which ultimately distributes to the satellite wells 112. In embodiments where 4 satellites wells 112 are used that may lead to four times (4×) less consumption of the sample.

A Third Immunoassay-Multiplexing Apparatus

According to further aspects of the present disclosure, another immunoassay-multiplex apparatus is disclosed. Unless stated otherwise, the references numbers hereinafter are analogous to the structures, definitions, embodiments, and variations thereof) of FIG. 3 except that the references numbers for the apparatus 500 are 200 numbers higher.

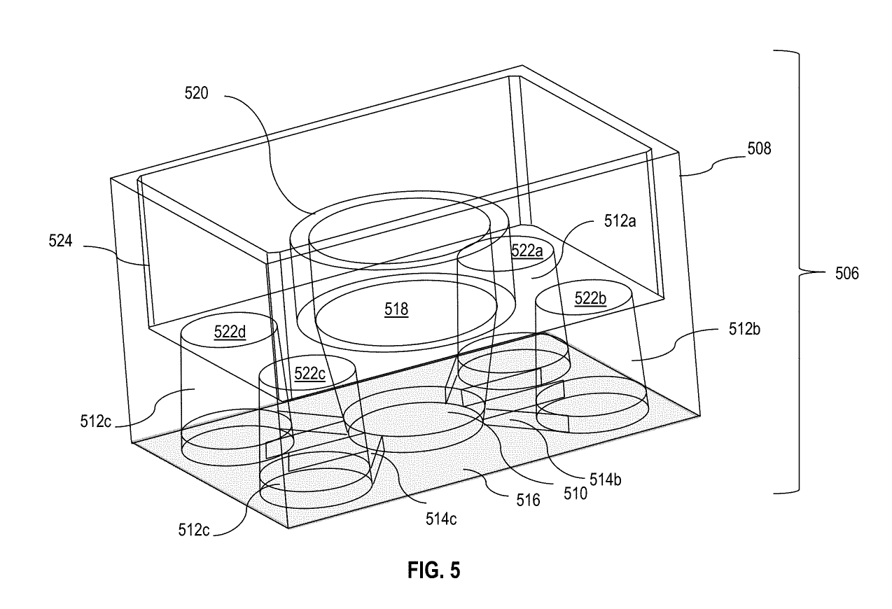

Referring to FIG. 5, the apparatus comprises a microplate having an array of cells. Each cell 506 comprises a container 508, wherein the container 508 has a chamber 524.

The container 508 further comprises a loading well 510 within the container 508 that accepts a sample of interest, the loading well 510 having an inferior inward taper, wherein the loading well 510 comprises a first frustro-conical portion defining the inferior inward taper.

Further, the container 506 comprises at least four satellite wells 512a, 512b, 512c, and 512d (collectively 512) arrayed around the loading well 510, wherein each satellite well 512 comprises a second frustro-conical portion defining a superior inward taper. In various embodiments, each of the satellite wells are equidistant from the loading well 510.

Yet further, the container 506 comprises least four interconnect channels 514b and 514c (514a and 514d not shown for clarity, collectively 514) that each independently couple the loading well 510 to an associated satellite well 512, wherein each interconnect channel 514 comprises an extruded trapezoidal shaped channel between the loading well 510 and its associated satellite well 512. Each interconnect channel 514 tapers inwardly as the interconnect channel 514 transitions from the associated satellite well 512 to the loading well 510.

With respect to the loading well 510, the satellite wells 512, and the interconnect channels 514, the first frustro-conical portion of the loading well 510 extends in a first dimension (i.e., downward in this embodiment). Further, the second frustro-conical portion of the satellite wells 514 extend oppositely in the first dimension spaced from the loading well 510 (i.e., upward in this embodiment, which is a taper opposite to the loading well 510). The satellite wells 512 accept identification markers through the second inlets 524, which interact with the sample of interest.

Moreover, the extruded trapezoidal shaped channel of the interconnect channel 514 extends in a second dimension orthogonal to the first dimension.

The chamber 520 extends over the first frustro-conical portion of the loading well 510 and defines a first inlet thereto and extends over the second frustro-conical portion of the satellite wells 512 and defines second inlets thereto.

The apparatus 500 further comprises a sealing layer 516 disposed on the bottom of the microplate. Analogously to the apparatus 500, the sealing layer 516 is a single piece that extends across the entire microplate.

Example Loading Procedure for an Immunoassay-Multiplexing Apparatus

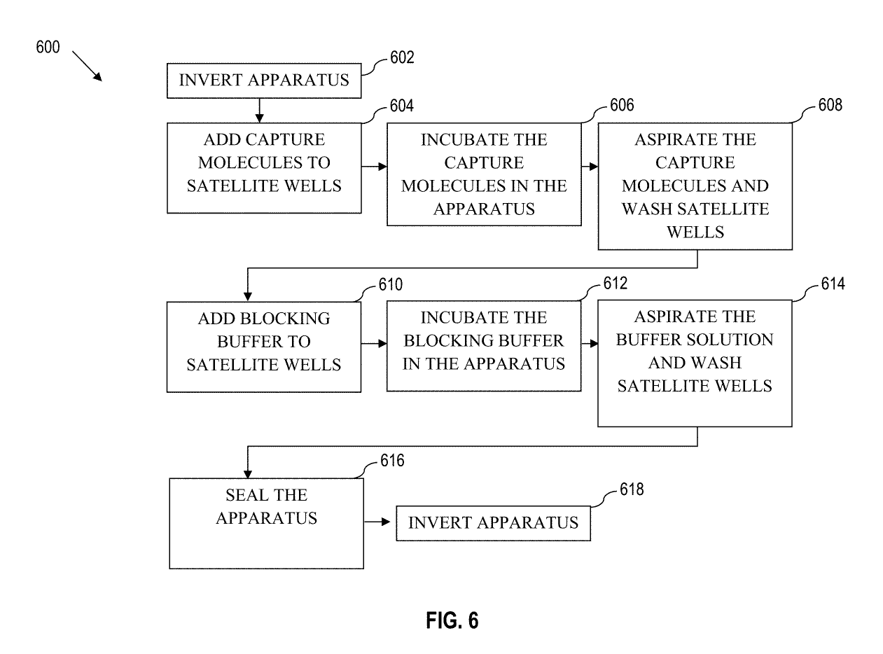

Referring to FIG. 6, a process 600 is provided for loading (e.g., pre-coating) an immunoassay-multiplexing apparatus. The process 600 can be utilized with certain implementations of the immunoassay-multiplexing apparatus embodiments described herein. The various embodiments and variations off the apparatuses of 100 and 300 are incorporated herein.

The process comprises inverting at 602 an immunoassay-multiplexing apparatus (hereinafter “apparatus”), wherein the apparatus comprises a microplate having an array of cells as described more fully herein.

For instance, as noted above, in certain embodiments, a microplate comprises an array of cells, where at least one cell within the array of cells comprises a container. Here, the container comprises a loading well having an inferior inward taper, a first satellite well within the container, a first interconnect channel that couples the loading well to the first satellite well, wherein the first interconnect channel tapers inwardly as the first interconnect channel transitions from the first satellite well to the loading well, a second satellite well within the container, and a second interconnect channel independent of the first interconnect channel that couples the loading well to the second satellite well, wherein the second interconnect channel tapers inwardly as the second interconnect channel transitions from the second satellite well to the loading well.

While the apparatus is inverted 602, the satellite well is oriented so that the inward taper is facing downward.

In various embodiments, the satellite wells may not have a top loading inlet. The top loading inlet is generally used to load various reagents into the satellite wells. However, in this case, reagents are being loaded from the bottom. Therefore, the top loading inlet is not needed. This type of embodiment may be useful in situations where the apparatus is pre-loaded with reagents prior to a customer or user buying the apparatus. Omitting the top loading inlet can reduce the potential of reagents entering the satellite wells and causing cross-reactivity when attempting to fill the loading well.

The process 600 further comprises adding at 604 capture molecules to the satellite wells. In some embodiments, 1 the capture molecules are prevented from entering the loading well by the inwardly taper of the interconnect channels. In various embodiments, the capture molecules are added only to fill the satellite wells so the capillary block action of the interconnect is not critical at this stage as the abrupt transition into the loading well creates a capillary barrier and the capture molecules are isolated in the satellite well and associated interconnect channel.

Once the capture molecules are added, the process 600 comprises incubating at 606 the capture molecules in the immunoassay-multiplexing apparatus for a predetermined amount of time. After incubation 606, the process 600 comprises aspirating and washing at 608 the capture molecules from the satellite wells (e.g., via an automated plate washer).

Moreover, the process 600 comprises adding at 610 a blocking buffer solution to the satellite wells. The blocking buffer blocks all the “unused” binding sites on the plastic surface not used by the capture molecules.

Once the blocking buffer is added, the process 600 comprises incubating at 612 the blocking buffer in the immunoassay-multiplexing apparatus for a predetermined amount of time. After incubation 606, the process 600 comprises aspirating and washing at 614 the blocking buffer from the satellite wells.

Thereinafter, the process 600 comprises sealing at 616 the apparatus with a hydrophilic sealing tape. The tape may be a singular piece, or in multiple parts (e.g., strips).

In addition, the process comprises inverting at 618 the immunoassay-multiplexing apparatus so that the immunoassay-multiplexing apparatus is returned to its default orientation (i.e., satellite well taper on the top end).

In various embodiments, it may be preferable to add a chamber to the apparatus. The chamber can be used as a space to wash the loading well and the satellite wells simultaneously, while the loading well is used to deliver the sample of interests, assays, reagents, etc. to the satellite wells. This configuration may be different from usual formats where the same “opening” to a given well is used to add/aspirate all the assay reagents, buffers and samples.

Analysis of Materials in the Immunoassay-Multiplexing Apparatus

Under traditional or conventional assay analysis, sample materials can be examined by using light, such as luminescence (e.g., fluorescence) and absorbance. Generally, an absorbance spectrophotometer is an instrument that measures the fraction of the incident light transmitted through a solution. In other words, it is used to measure the amount of light that passes through a sample material and, by comparison to the initial intensity of light reaching the sample, they indirectly measure the amount of light absorbed by that sample.

However, such traditional and conventional techniques have inherent flaws. For instance, most sample materials are held in some sort of a container while being analyzed by absorbance. As a result, materials that comprise the container can skew an amount of light ultimately received by the absorbance spectrophotometer (i.e., light or illumination noise caused by the container), which may lead to a miscalculation (i.e., incorrect value) in an absorbance value of the sample material.

To facilitate reducing, or even eliminating the flaws above, aspects of the present disclosure provide for utilizing two wavelengths to create an illumination silent noise mask, which cancels out the illumination noise caused by the materials of the container.

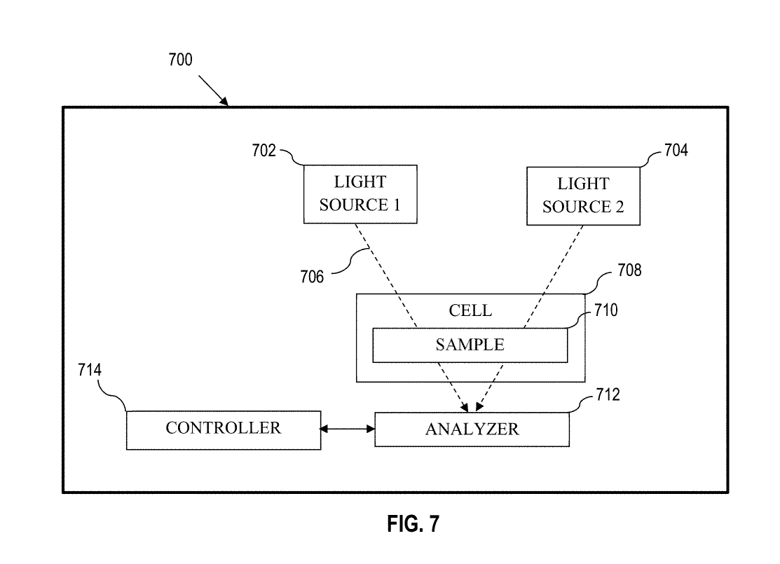

Referring to FIG. 7, an example set-up 700 for immuno-assay analysis using two wavelengths to create the illumination silent noise mask is illustrated. In this example, the set-up 700 includes a first light source 702 and a second light source 704, each of which emit light 706 (or a “wavelength”) through a cell 708 (e.g., the cell of FIG. 1, ref number 106) containing a sample material 710. The light 706 passes through the cell 708 and sample material 710 to an analyzer 712 (e.g., absorbance spectrophotometer) that is connected to a controller 714 to manage, process, and control function of associated hardware.

The illumination silent nose mask can be created through various techniques. For example, a first wavelength projects from the first light source 702 through a blank cell that does not have the sample material within to the analyzer 712, thus establishing a baseline absorbance for the cell 708. The second light source 704 projects a second wavelength through the cell 708 containing the sample material 710, to the analyzer 712 for measurement. The baseline absorbance for the cell 708 can then be factored out of the measurement by the second light source 704 and analyzer 712, thus resulting in an accurate absorbance reading absent illumination noise (i.e., illumination silent noise mask).

In various embodiments, the first light source 702 and the second light source 704 project in sequence. In other embodiments, the first light source 702 and the second light source 704 project in parallel.

A further consideration when analyzing immune-assays apparatuses with multiple sample spaces being fed from a singular source is unequal distribution into the sample spaces (e.g., the four satellite wells receiving reagents from the loading well, see FIG. 5). In a scenario with four satellite wells, ideally each satellite well would take 25% of the reagent that is introduced into the loading well. However, practical realities undercut that ideal. In this regard, different concentrations of the reagent in each satellite well will ultimately affect absorbance values.

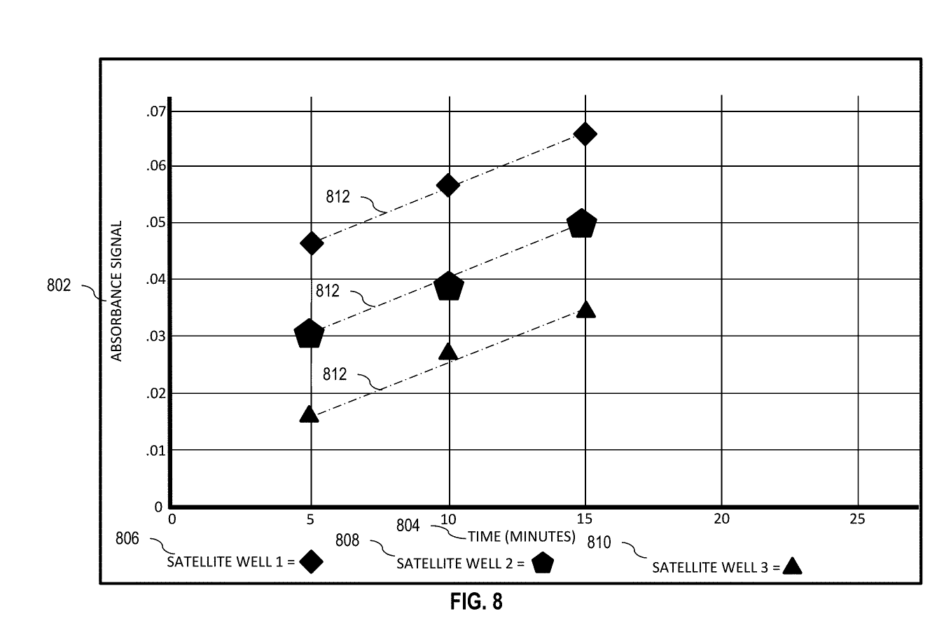

Accordingly, aspects of the present disclosure allow for accurate measurement of each satellite well despite the relative or different concentrations of reagent within each satellite well by calculating a slope for each satellite well as illustrated in FIG. 8.

FIG. 8 illustrates a graph 800 of a slope for three satellite wells that were fed by the same loading well and measured by absorbance 802 three times over a time 804 period of fifteen minutes. A first satellite well (“Satellite Well 1”) 806 is symbolized by a diamond, a second satellite well (“Satellite Well 2”) 808 is symbolized by a pentagon, and a third satellite well (“Satellite Well 3”) 810 is symbolized by a triangle. The following measurements are for illustrative purposes and are by no means limiting.

In this example, the first satellite 806 well received the most reagent, the second satellite well 808 received less reagent than the first satellite well 806, and the third satellite well 810 received less reagent than both the first satellite well 806 and the second satellite well 808.

As illustrated in the graph 800, at five minutes the absorbance of the first satellite well 806 is approximately 0.0175, the absorbance of the second satellite well 808 is approximately 0.0300, and the absorbance of the third satellite well 810 is approximately 0.0475.

As further illustrated in the graph 800, at ten minutes the absorbance of the first satellite well 806 is approximately 0.0280, the absorbance of the second satellite well 808 is approximately 0.0390, and the absorbance of the third satellite well 810 is approximately 0.0575.

As yet further illustrated in the graph 800, at fifteen minutes the absorbance of the first satellite well 806 is approximately 0.0340, the absorbance of the second satellite well 808 is approximately 0.0500, and the absorbance of the third satellite well 810 is approximately 0.0655.

When a slope 812 is calculated for each satellite well (e.g., y=mx+b) as shown in the graph 800, the slope for each well is unexpectantly consistent despite the disproportionate concentration of reagent distributed to each satellite well. Further, slopes 812 can be combined with the technique of using two wavelengths to increase reliability and accuracy of data measurements of the sample materials in the satellite wells.

While the previous examples and disclosures herein have focused on absorbance, it is by no means the only metric or measurement that can be practiced or implemented with aspects of the present disclosure.

A Process for Analyzing an Immunoassay-Multiplexing Apparatus

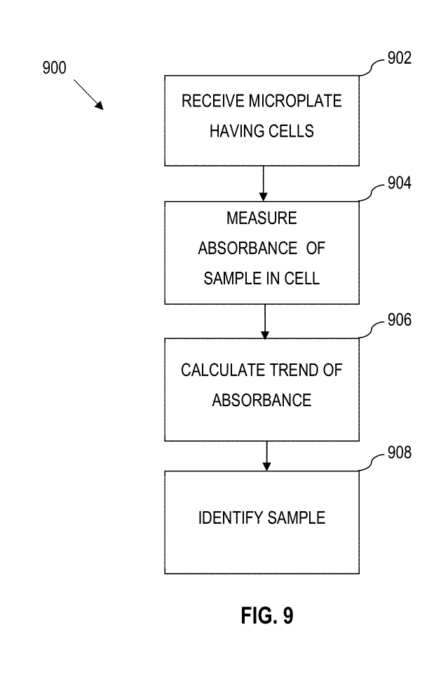

Referring to the figures, and in particular FIG. 9 a process 900 for analyzing an immunoassay-multiplexing apparatus is disclosed.

The process 900 comprises receiving at 902 a microplate having an array of cells. The array of cells can be similar to those disclosed herein (or a variation thereof). For instance, as noted above, in certain embodiments, a microplate comprises an array of cells, where at least one cell within the array of cells comprises a container. Here, the container comprises a loading well having an inferior inward taper, a first satellite well within the container, a first interconnect channel that couples the loading well to the first satellite well, wherein the first interconnect channel tapers inwardly as the first interconnect channel transitions from the first satellite well to the loading well, a second satellite well within the container, and a second interconnect channel independent of the first interconnect channel that couples the loading well to the second satellite well, wherein the second interconnect channel tapers inwardly as the second interconnect channel transitions from the second satellite well to the loading well. The cell also comprises a sealing layer, e.g., of a hydrophilic sealing tape, disposed on the bottom of the microplate.

Moreover, the process comprises loading at 904 a sample material into the loading well, thereby distributing the sample material into the plurality of satellite wells via the interconnect channel, wherein the inwardly tapered interconnect channel prevents the sample material from re-entering the loading well.

The process 900 further comprises measuring at 906 an absorbance signal of a sample material in each satellite well within the plurality of satellite wells, at predetermined time intervals (e.g., at 5/10/15 minutes, 10/20/30 minutes, etc., see FIG. 8 and related disclosure thereto).

Moreover, the process 900 further comprises calculating at 908 a trend based on the measured absorbance signal and time intervals. For instance, the trend can be a slope as described in the disclosure and explanation of the graph 800 herein.

In addition, the process 900 comprises identifying at 910 the sample material based on the trend. For example, sample materials can be identified 910 by comparing the absorbance (based on the trend) to a library of known sample materials or by utilizing other data sources.

Example Workflow

The following is an example workflow using an immunoassay-multiplexing apparatus as described herein using a sandwich format. After receiving a microplate having a satellite wells (e.g., the microplate 102 in FIGS. 1A-1D) that are precoated with a primary antibody, a sample material is added to the loading well and incubated. After incubation, the sample material is aspirated (removed), and a first wash buffer is added to the loading well and then aspirated.

Further, a detection anti-body with an enzyme is added to the loading well and incubated. After incubation, the detection anti-body is aspirated, and a second wash buffer (which may be the same as or different than the first wash buffer) is added to the loading well and then aspirated.

Thereafter, a predetermined amount of absorbance substrate is added to the loading well such that the volume is divided to the satellite wells as described herein. Absorption of the sample in the cells is measured, and a trend is calculated as described in greater detail herein, and the sample is identified based on the calculated trend.

Miscellaneous Considerations

The various immunoassay-multiplexing apparatus configurations described more fully herein can be configured to work with commonly used pipetors, liquid handling systems and widely used absorbance microplate readers (all of which are already available in labs that run conventional ELISA).

Moreover, the specific design of the apparatus as described more fully herein, provides a geometry-driven capillary force control that enables accurate, passive flow regulation for assay sequencing. As such, the apparatus allows any lab that runs ELISA's to run x-plex multiplexing with the same protocol as singleplex ELISAs (where x is the number of satellite wells, e.g., four satellite wells achieves 4-plex, etc.). That is, geometry-directed capillary force control enables the flow control for the apparatus herein. That is, geometrical design is used to manipulate the meniscus of a liquid column thereby achieving targeted flow.

The meniscus of a liquid-solid interface is dictated by the liquid type and the surface energy of the solid it is in contact with. As noted more fully herein, the loading well and satellite wells have opposing tapers. This leads to different menisci and a differential capillary force resulting in capillary flow from the loading well to the satellite well(s).

When the loading well is connected to a satellite well by the described interconnect channel (e.g., a microfluidic interconnect), the geometrically-directed meniscus control forces a weak hydrophobic meniscus in the load well and a strongly hydrophilic interface in the satellite well thereby forcing the liquid from the loading to the satellite wells. As the liquid reaches the top of the satellite well; significant energy (differential pressure) is required for the liquid to exit the well and an equilibrium condition is reached.

Compared to 96-well assays, the apparatus herein offers significantly higher capacity (e.g., 256 data points/run in the apparatus 100 of FIG. 5 using cells with four satellite wells) compared to 96-well assays. Moreover, assays using the apparatus herein require far less effort (or higher throughput for the same number of steps).

The 96-well like liquid handling interface can also be configured to be compatible with current automated liquid handlers and labs using automated liquid handlers can likely achieve considerable throughput gains. Compared to 384-well assays, with a 96-well like liquid handling interface, the apparatus herein can be operated manually whereas 384-well plates require automated liquid handlers.

The apparatus described herein can be compatible with all commonly used assay formats such as sandwich, competitive and indirect assay. Note that for automated liquid handling, column increments will be customized (easy to modify in program script) for both the 96-well interface as well as the 384-well interface since some columns are “skipped”.

The physical design of the apparatus ensures that liquid added to the loading well is then evenly directed to each of the satellite wells and the excess remains in the loading well. This ensures that the satellite wells are completely filled to ensure consistent performance. For example, as a liquid substrate empties out of the loading well, the fluidic connection between the satellite wells is broken thereby eliminating the possibility of cross-talk due to diffusion between adjacent wells. After substrate development, the plate is then read as a 384-well plate (also easily programmed to read selective columns).

According to aspects herein, the apparatus addresses some of the disadvantages of current solutions including: (a) no need for capital investment; (b) similar operating procedure as 96-well plate ELISA and no additional training; and (c) reduced susceptibility to rheumatoid/HAMA interference with planar multiplexing format. Two benefits of the apparatus over current multiplexing include: (a) compatibility with all assay formats and (b) potential to eliminate the cross-reactivity problem.

The overall shape of the satellite wells can be driven by the intended purpose of a given apparatus. For instance, where factors such as assay sensitivity, volume variation, evaporation, or differential pressure are of importance, a conical design is selected. Where assay sensitivity is a driving factor, the top diameter of a corresponding satellite well may be much smaller than its corresponding base diameter. For a factor such as absorbance, then a cylindrical design can be utilized to maximize signal strength.

Microfluidic channels can be susceptible to blockage due to particulates in biological matrices. For example, plasma samples centrifuged at lower speeds (or collected from precipitated whole blood) are likely to have some cellular fraction. Microfluidic channels of smaller dimensions can be blocked if these cellular fractions are not filtered out. As such the interconnect channels 114 can be dimensioned as ˜200 μm×200 μm (or higher) in order to offer reasonable performance with biological matrices and small bubbles. As such, even difficult to process matrices such as tissue homogenate extracts can be processed by the apparatus herein.

In certain embodiments, as liquid (capture molecule solution) is added to the satellite well, the liquid will fill the satellite well as well as the microfluidic interconnect since the microfluidic interconnect is sealed on one side by a strongly hydrophilic adhesive tape. The hydrophilic tape also helps in creating a uni-directional capillary stop at the interface between the interconnect and the loading well. As the liquid fills the interconnect (from satellite well) and approaches the loading well, the dimension of the flow path abruptly transitions due to the interconnect channel design and creates a capillary stop so that the liquid does not flow into the loading well. On the other hand, when liquid is added to the loading well and meets the microfluidic interconnect interface, the outward taper (away from loading well) serves to reduce the curvature of the liquid interface. However, owing to the small depth, the meniscus shape across the depth dominates the flow behavior and the liquid will be drawn into the microfluidic interconnect.

As the liquid now approaches the satellite well via the interconnect, the width of the interconnect expands to match the base diameter of the satellite well. This allows the liquid to form a meniscus at the base of the satellite well. As the satellite well tapers upwards (and with the differential capillary force across the liquid plug with a very weakly hydrophilic (tending to hydrophobic) meniscus in the loading well) the liquid can now fill the satellite well. In this regard, the loading well taper can be specifically designed to ensure a higher differential pressure between the loading and satellite wells. The lower limit constraint for the base diameter is the width of the microfluidic interconnect. The base of the loading well should ensure that the interconnects are separated to create the capillary stop effect. The top diameter of the loading well can be as large as possible to ensure easy manual operation. Moreover, in at least one embodiment, the volume of the loading well should be at least 20% greater than the volume of the satellite well(s).

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. Aspects of the disclosure were chosen and described in order to best explain the principles of the disclosure and the practical application, and to enable others of ordinary skill in the art to understand the disclosure for various embodiments with various modifications as are suited to the particular use contemplated.

What is claimed is:

1. An immunoassay-multiplexing apparatus comprising:
a microplate comprising an array of cells, wherein a select cell within the array of cells comprises:
a container comprising:
a loading well within the container having an inferior inward taper;
a satellite well within the container; and
an interconnect channel that couples the loading well to the satellite well, wherein the interconnect channel tapers inwardly as the interconnect channel transitions from the satellite well to the loading well; and
a sealing member that seals the cell.

2. The immunoassay-multiplexing apparatus of claim 1, wherein the satellite well defines a first satellite well and the interconnect channel further defines a first interconnect channel, the container further comprising:
a second satellite well within the container; and
a second interconnect channel that couples the loading well to the second satellite well, wherein the second interconnect channel tapers inwardly as the second interconnect channel transitions from the second satellite well to the loading well.

3. The immunoassay-multiplexing apparatus of claim 2, wherein:
the first satellite well and the second satellite well are equidistant from the loading well.

4. The immunoassay-multiplexing apparatus of claim 2, wherein:
the interconnect channel of the first satellite well and/or the second satellite well further comprises an indent proximally located where the interconnect channel and the loading well meet.

5. The immunoassay-multiplexing apparatus of claim 2 further comprising:
a third satellite well within the container;
a third interconnect channel that couples the loading well to the third satellite well, wherein the third interconnect channel tapers inwardly as the third interconnect channel transitions from the third satellite well to the loading well;
a fourth satellite well within the container; and
a fourth interconnect channel that couples the loading well to the fourth satellite well, wherein the fourth interconnect channel tapers inwardly as the fourth interconnect channel transitions from the fourth satellite well to the loading well.

6. The immunoassay-multiplexing apparatus of claim 5, wherein:
the first satellite well, the second satellite well, the third satellite well, and the fourth satellite well are each equidistant from the loading well.

7. The immunoassay-multiplexing apparatus of claim 1, wherein:
the container further comprises a chamber disposed above the satellite well, the chamber defining a first inlet for the loading well and a second inlet for the satellite well.

8. The immunoassay-multiplexing apparatus of claim 1, wherein:
the microplate comprises a microplate frame where the loading well is oriented to conform to a conventional 96-well form factor in at least one direction.

9. The immunoassay-multiplexing apparatus of claim 1, wherein:
the microplate comprises a microplate frame where the loading well is oriented to conform to a conventional 384-cell form factor and/or a conventional 1536-well form factor in at least one direction.

10. The immunoassay-multiplexing apparatus of claim 1, wherein:
the container further comprises a chamber;
the loading well comprises a first frustro-conical portion defining the inferior inward taper;
the satellite well comprises a second frustro-conical portion defining a superior inward taper;
the interconnect channel comprises an extruded trapezoidal shaped channel between the loading well and the satellite well;
wherein:
the first frustro-conical portion of the loading well extends in a first dimension;
the second frustro-conical portion of the satellite well extends oppositely in the first dimension spaced from the loading well;
the extruded trapezoidal shaped channel of the interconnect channel extends in a second dimension orthogonal to the first dimension;
the chamber extends over the first frustro-conical portion of the loading well and defines a first inlet thereto; and
the chamber extends over the second frustro-conical portion of the satellite well and defines a second inlet thereto.

11. The immunoassay-multiplexing apparatus of claim 1, wherein:
the satellite well has a superior inward taper.

12. The immunoassay-multiplexing apparatus of claim 1, wherein:
the interconnect channel that tapers inwardly as the interconnect channel transitions from the satellite well to the loading well has an extruded trapezoidal shaped geometry.

13. The immunoassay-multiplexing apparatus of claim 1, wherein:
the sealing member is a hydrophilic sealing member.

14. The immunoassay-multiplexing apparatus of claim 1, wherein:
the cells within the array of cells are comprised of at least one of a polystyrene, polycarbonate, Polymethyl methacrylate (PMMA), and cyclic olefin copolymers (COC).

15. The immunoassay-multiplexing apparatus of claim 1, wherein:
the cells within the array of cells are comprised of a hydrophobic polystyrene material.

16. The immunoassay-multiplexing apparatus of claim 1, wherein:
the cells within the array of cells are arranged in a strip-well format such that multiple strip-wells can be assembled within a frame of the microplate.

17. The immunoassay-multiplexing apparatus of claim 16, wherein:
the microplate further comprises tab members that accept the cells within the array of cells are arranged in the strip-well format.

18. A process for loading an immunoassay-multiplexing apparatus, the process comprising:
inverting an immunoassay-multiplexing apparatus, the immunoassay-multiplexing apparatus comprising:
a microplate comprising an array of cells, wherein at least one cell within the array of cells comprises:
a container comprising:
a loading well having an inferior inward taper;
a first satellite well within the container;
a first interconnect channel that couples the loading well to the first satellite well, wherein the first interconnect channel tapers inwardly as the first interconnect channel transitions from the first satellite well to the loading well;
a second satellite well within the container; and
a second interconnect channel independent of the first interconnect channel that couples the loading well to the second satellite well, wherein the second interconnect channel tapers inwardly as the second interconnect channel transitions from the second satellite well to the loading well;
adding capture molecules to both the first satellite well and the second satellite well;
incubating the capture molecules in the immunoassay-multiplexing apparatus for a predetermined amount of time;
aspirating the capture molecules, and then washing the first satellite well and the second satellite well;
adding a blocking buffer solution to the first satellite well and the second satellite well;
incubating the blocking buffer in the immunoassay-multiplexing apparatus for a predetermined amount of time;
aspirating the blocking buffer solution, and then washing the first satellite well and the second satellite well;
inverting the immunoassay-multiplexing apparatus so that the immunoassay-multiplexing apparatus is returned to its default orientation; and
sealing the immunoassay-multiplexing apparatus with a sealing layer.

19. A process for analyzing an immunoassay-multiplexing apparatus comprising:
receiving a microplate comprising an array of cells, wherein at least one cell within the array of cells comprises:
a container comprising:
a loading well having an inferior inward taper;
a first satellite well within the container;
a first interconnect channel that couples the loading well to the first satellite well, wherein the first interconnect channel tapers inwardly as the first interconnect channel transitions from the first satellite well to the loading well;
a second satellite well within the container;
a second interconnect channel independent of the first interconnect channel that couples the loading well to the second satellite well, wherein the second interconnect channel tapers inwardly as the second interconnect channel transitions from the second satellite well to the loading well; and
a sealing layer disposed on the bottom of the microplate;
loading a sample material into the loading well, thereby distributing the sample material into the first satellite well via the first interconnect channel, and into the second satellite well via the second interconnect channel, wherein the inward taper of the first interconnect channel prevents the sample material from re-entering the loading well from the first satellite well, and the inward taper of the second interconnect channel prevents the sample material from re-entering the loading well from the second satellite well;
measuring an absorbance signal of the sample material in each of the first satellite well and the second satellite well at predetermined time intervals;
calculating a trend based on the measured absorbance signal and time intervals; and
identifying the sample material based on the trend.
